(19) United States
(12) Patent Application Publication
(10) Pub. No.: US 2024/0408006 A1
(43) Pub. Date: Dec. 12, 2024

(54) LEVOTHYROXINE COMPOSITIONS AND IT'S PROCESS

(71) Applicant: Neuheit Pharma Technologies Pvt. Ltd, Hyderabad (IN)

(72) Inventors: Venkateswarlu Vobalaboina, Hyderabad (IN); Srinivas Irukulla, Hyderabad (IN); Vamshi Krishna Vobalaboina, Hyderabad (IN)

(21) Appl. No.: 18/684,135

(22) PCT Filed: Aug. 16, 2022
(86) PCT No.: PCT/IB22/57646

§ 371 Date: Feb. 15, 2024

(30) Foreign Application Priority Data
Aug. 19, 2021 (IN) 202141037676

(51) Int. Cl. A61K 9/00; A61K 9/20; A61K 31/198

(52) U.S. Cl. CPC A61K 9/006; A61K 9/2018; A61K 9/2054; A61K 9/2059; A61K 31/198

(57) ABSTRACT

The present invention relates to Levothyroxine compositions and its process for preparation. The present invention specifically relates to a composition of Levothyroxine sublingual tablets comprising Levothyroxine or its pharmaceutically acceptable salts and pharmaceutically acceptable excipients. The present invention more specifically relates to a composition of Levothyroxine sublingual tablets comprising Levothyroxine or its pharmaceutically acceptable salts and pharmaceutically acceptable excipients selected from fillers/diluents, superdisintegrants, antioxidants, surfactants, stabilizers, buffering agents, lubricants and solvents. The present invention also relates to a process for the preparation of Levothyroxine sublingual tablets by direct compression method or granulation method.

### FIELD OF INVENTION

The present invention relates to Levothyroxine compositions and its process for preparation.

The present invention specifically relates to a composition of Levothyroxine sublingual tablets comprising Levothyroxine or its pharmaceutically acceptable salts and pharmaceutically acceptable excipients.

The present invention more specifically relates to a composition of Levothyroxine sublingual tablets comprising Levothyroxine or its pharmaceutically acceptable salts and pharmaceutically acceptable excipients selected from fillers/diluents, superdisintegrants, antioxidants, surfactants, stabilizers, buffering agents, lubricants and solvents.

The present invention also relates to a process for the preparation of Levothyroxine sublingual tablets by direct compression method or granulation method.

### BACKGROUND OF INVENTION

Thyroid hormones are two hormones produced and released by the thyroid gland, namely triiodothyronine (T₃) and thyroxine (T₄). They are tyrosine-based hormones that are primarily responsible for regulation of metabolism. T₃ and T₄ are partially composed of iodine. A deficiency of iodine leads to decreased production of T₃ and T₄, enlarges the thyroid tissue and will cause the disease known as simple goiter. The major form of thyroid hormone in the blood is thyroxine (T₄), which has a longer half-life than T₃.

Levothyroxine, also known as L-thyroxine, is a manufactured form of the thyroid hormone thyroxine (T₄). It is used to treat thyroid hormone deficiency (hypothyroidism), including Hashimoto's disease and a severe form known as myxedema coma. It may also be used to treat and prevent certain types of thyroid tumors. It is not indicated for weight loss. Levothyroxine is taken by mouth or given by intravenous injection. Maximum effect from a specific dose can take up to six weeks to occur.

Levothyroxine, a synthetic crystalline levothyroxine (T4) in sodium salt form. Synthetic T₄ is identical in chemical structure to the T4 produced in the human thyroid gland. The chemical name of Levothyroxine sodium is (2S)-2-amino-3-[4-(4-hydroxy-3,5-diiodophenoxy)-3,5-diiodophenyl]propanoate. Levothyroxine sodium has a chemical formula of C₁₅H₁₀I₄NNaO₄·xH₂O and a molecular mass of 798.85 g/mol (anhydrous). It has a structural formula of:

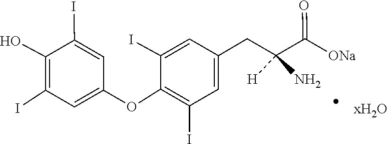

Sublingual tablets promote rapid absorption and higher bioavailability with an almost instant onset of action. A sublingual tablet designed to promote the retention of the active drug substance under the tongue, to prevent its swallowing, and to minimize inter and intra individual variability. Sublingual tablets manufactured by the direct compression method exhibit good mechanical strength and acceptably fast disintegration.

U.S. Pat. No. 6,555,581 B1 discloses stable, solid, immediate release pharmaceutical tablet for oral consumption comprising 0.01 mg to about 0.8 mg levothyroxine sodium, 100 mg to about 110 mg of β-microcrystalline cellulose particles, 25 mg to about 50 mg of croscarmellose sodium and 0.5 mg to about 5 mg of magnesium stearate.

U.S. Pat. No. 6,872,405 B2 discloses quick-disintegrating tablet in the buccal cavity comprising a drug, a diluent, and a saccharide with a relatively lower melting point than the drug and the diluent, which is obtained by uniformly mixing the saccharide with a low melting point in the tablet so that a bridge will be formed between said drug and/or said diluent particles by the product of melting and then solidification of this saccharide with a low melting point.

U.S. Pat. No. 6,936,274 B2 discloses storage-stable dosage form of a thyroxine active drug composition which exhibits an improved stability. The formulation contains a thyroxine active drug substance, an alditol, and a saccharide, and, optionally, additional pharmaceutically accepted excipients.

U.S. Pat. No. 8,545,881 B2 discloses tablet that rapidly disintegrates in the oral cavity comprising a compressed blend of rapidly dispersing microgranules prepared by granulating a sugar alcohol or a saccharide or a mixture thereof having an average particle size less than about 30 microns and a disintegrant.

U.S. Pat. No. 9,682,045 B2 discloses stable pharmaceutical composition comprising a thyroid hormone drug or pharmaceutically acceptable salts thereof, at least one carbohydrate, wherein the carbohydrate is a saccharide, and one or more pharmaceutically acceptable excipients, wherein the composition retains at least 95% of the potency of levothyroxine sodium after storage for 24 months at 25° C. and 75% relative humidity.

On the contrary, the inventors of the present application have found the composition of Levothyroxine sublingual tablets comprising Levothyroxine or its pharmaceutically acceptable salts and pharmaceutically acceptable excipients selected from fillers/diluents, superdisintegrants, antioxidants, surfactants, binding agents, buffering agents, lubricants and solvents. The present invention also relates to process for the preparation of Levothyroxine sublingual tablet by direct compression method or granulation method.

The inventors of the present application have surprisingly found that the Levothyroxine sublingual tablets of the present invention are physicochemically stable and having good dissolution profiles.

### Objective of Invention

The main objective of the present invention is to provide Levothyroxine compositions and its process for preparation.

Another objective of the present invention is to provide a composition of Levothyroxine sublingual tablet comprising Levothyroxine or its pharmaceutically acceptable salts and pharmaceutically acceptable excipients.

Another objective of the present invention is to provide a composition of Levothyroxine sublingual tablets comprising Levothyroxine or its pharmaceutically acceptable salts and pharmaceutically acceptable excipients selected from fillers/diluents, superdisintegrants, antioxidants, surfactants, stabilizers, buffering agents, lubricants and solvents.

Still another objective of the present invention is to provide a process for the preparation of Levothyroxine sublingual tablet by direct compression method or granulation method.

### SUMMARY OF INVENTION

Accordingly, the present invention provides Levothyroxine compositions and its process for preparation.

One embodiment of the present invention provides a composition of Levothyroxine sublingual tablet comprising Levothyroxine or its pharmaceutically acceptable salts and pharmaceutically acceptable excipients.

Another embodiment of the present invention provides a composition of Levothyroxine sublingual tablets comprising Levothyroxine or its pharmaceutically acceptable salts and pharmaceutically acceptable excipients selected from fillers/diluents, superdisintegrants, antioxidants, surfactants, stabilizers, buffering agents, lubricants and solvents.

Another embodiment of the present invention provides a composition of sublingual tablet comprising Levothyroxine sodium as active ingredient, mannitol or combination of mannitol and starch, microcrystalline cellulose and silicified microcrystalline cellulose or combinations thereof as fillers/diluents, croscarmellose sodium as superdisintegrant, butylated hydroxyanisole as antioxidant, sodium lauryl sulfate or glyceryl monostearate as surfactant, sodium alginate as stabilizer, sodium bicarbonate as buffering agent, magnesium stearate as lubricant.

Yet another embodiment of the present invention provides a sublingual tablet composition comprising:
a) 0.01% to 3% w/w of active ingredient,
b) 65% to 95% w/w of fillers or diluents,
c) 1% to 15% w/w of superdisintegrants,
d) 0.1% to 3% w/w of antioxidants,
e) 0.1% to 3% w/w of surfactants,
f) 1% to 5% w/w of stabilizers,
g) 0.1% to 3% w/w of lubricant, and optionally
h) 0.1% to 10% w/w of other pharmaceutically acceptable excipients.

Yet another embodiment of the present invention provides a sublingual tablet composition comprising:
a) 0.01% to 3% w/w of Levothyroxine sodium,
b) 65% to 95% w/w of fillers or diluents selected from mannitol or combination of mannitol and starch, microcrystalline cellulose and silicified microcrystalline cellulose or combinations thereof,
c) 1% to 15% w/w of croscarmellose sodium,
d) 0.1% to 3% w/w of butylated hydroxyanisole,
e) 0.1% to 3% w/w of sodium lauryl sulfate or glyceryl monostearate,
f) 1% to 5% w/w of sodium alginate,
g) 0.1% to 3% w/w of magnesium stearate and optionally
h) 0.1% to 10% w/w of other pharmaceutically acceptable excipients.

Yet another embodiment of the present of the present invention is to provide a process for the preparation of Levothyroxine sodium sublingual tablet by direct compression method or granulation method.

In yet another embodiment, the present invention provides process for preparing sublingual tablet, the process comprising steps of:
a) mixing active ingredient geometrically with buffering agent and fillers/diluents,
b) mixing obtained blend with other excipients,
c) lubricating the obtained blend with lubricant,
d) compressing the lubricated blend into tablet, and
e) packing obtained sublingual tablet.

In yet another embodiment, the present invention provides process for preparing sublingual tablet, the process comprising steps of:
a) mixing active ingredient geometrically with surfactant and antioxidant,
b) mixing obtained blend with superdisintegrant and then with fillers/diluents,
c) adding stabilizing, fillers/diluents to step (b) and blending for 5-10 min using rapid mixer granulator (RMG),
d) lubricating the obtained blend with lubricant for 5-10 min,
e) compressing the lubricated blend into tablet, and
f) packing obtained sublingual tablet.

In yet another embodiment, the present invention provides process for preparing sublingual tablet, the process comprising steps of:
a) mixing active ingredient geometrically with surfactant and antioxidant,
b) mixing obtained blend with superdisintegrant and then with fillers/diluents,
c) granulating the obtained blend with solvent using RMG or Fluidized Bed Coater (FBC) followed by drying in Fluidized bed dryer (FBD),
d) adding stabilizing agent, fillers/diluents and blending for 5-10 min using rapid mixer granulator (RMG),
e) lubricating the obtained blend with lubricant for 5-10 min,
f) compressing the lubricated blend into, and
g) packing obtained sublingual tablet in HDPE bottle or blister pack.

In yet another embodiment, the present invention provides a process for preparing Levothyroxine sodium sublingual tablet, the process comprising steps of:
a) mixing Levothyroxine sodium geometrically with sodium lauryl sulfate or glyceryl monostearate and butylated hydroxyanisole,
b) mixing obtained blend with croscarmellose sodium and then with combination of mannitol and starch,
c) adding sodium alginate, microcrystalline cellulose and blending for 5-10 min using rapid mixer granulator (RMG),
d) lubricating the obtained blend with magnesium Stearate for 5-10 min, e) compressing the lubricated blend into tablet, and
f) packing obtained Levothyroxine sublingual tablet in HDPE bottle or blister pack.

In yet another embodiment, the present invention provides process for preparing levothyroxine sodium sublingual tablet, the process comprising steps of:
a) mixing Levothyroxine sodium geometrically with sodium lauryl sulfate or glyceryl monostearate and butylated hydroxyanisole,
b) mixing obtained blend with croscarmellose sodium and then with mannitol,
c) granulating the obtained blend with absolute alcohol using RMG or Fluidized Bed Coater (FBC) followed by drying in Fluidized bed dryer (FBD),
d) adding sodium alginate, microcrystalline cellulose and blending for 5-10 min using rapid mixer granulator (RMG),
e) lubricating the obtained blend with magnesium Stearate for 5-10 min,
f) compressing the lubricated blend into tablet, and
g) packing obtained Levothyroxine sublingual tablet in HDPE or blister pack.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The present invention provides composition of thyroid hormone sublingual tablet comprising Levothyroxine sodium as active ingredient and pharmaceutically acceptable excipients.

The concentration of active ingredient used in the sublingual tablet is from 0.01% to 3% (w/w), more preferably 0.01% to 2% (w/w) of the total weight of the composition.

Fillers or diluents used in the present invention are selected from and not limited to mannitol, Pearlitol flash (co-processed mannitol and starch), microcrystalline cellulose, silicified microcrystalline cellulose, dibasic calcium phosphate, powdered cellulose, tribasic calcium phosphate, calcium carbonate, calcium sulfate, dextran, dextrin, dextrose, fructose, kaolin, lactose, sorbitol, starch, pregelatinized starch, sucrose, xylitol and lactose. Preferably used fillers or diluents are mannitol or Pearlitol flash (co-processed mannitol and starch), microcrystalline cellulose and silicified microcrystalline cellulose or combinations thereof.

The concentration of fillers or diluents used in the sublingual tablet is from 65% to 95% (w/w), more preferably 70% to 95% (w/w) of the total weight of the composition.

Superdisintegrants used in the present invention are selected from and not limited to sodium starch glycolate and croscarmellose sodium. Preferably used superdisintegrant is croscarmellose sodium.

The concentration of superdisintegrant used in the sublingual tablet is from 1% to 15% (w/w), more preferably 1% to 13% (w/w) of the total weight of the composition.

Antioxidants used in the present invention are selected from and not limited to ascorbic acid, citric acid, ascovir palmitate, monothioglycerol, butylated hydroxyanisole, butylated hydroxytoluene (BHT), potassium metabisulfite, propyl gallate and tocopherol. Preferably used antioxidant is butylated hydroxyanisole.

The concentration of antioxidant used in the sublingual tablet is from 0.1% to 3% (w/w), more preferably 0.1% to 2% (w/w) of the total weight of the composition.

Surfactants used in the present invention are selected from and not limited to sodium lauryl sulfate, glyceryl monostearate and poloxamers (polyoxyethylene and polyoxypropylene copolymers), natural or synthetic lecithin, esters of sorbitan and fatty acids. Preferably used surfactant is sodium lauryl sulfate or glyceryl monostearate.

The concentration of surfactants used in the sublingual tablet is from 0.1% to 3% (w/w), more preferably 0.1% to 2% (w/w) of the total weight of the composition.

Stabilizers used in the present invention are selected from and not limited to, sodium alginate, agar, alginic acid and alginates, carrageenan calcium. Preferably used Stabilizer is sodium alginate.

The concentration of stabilizers used in the sublingual tablet is from 1% to 5% (w/w), more preferably 2% to 5% (w/w) of the total weight of the composition.

Buffering agents used in the present invention are selected from and not limited to sodium bicarbonate, calcium carbonate, calcium formate, magnesium hydroxide, aluminum, aluminum hydroxide/magnesium hydroxide co-precipitate, aluminum hydroxide/sodium bicarbonate co-precipitate, calcium acetate, calcium bicarbonate, calcium borate, calcium bicarbonate, calcium citrate, calcium gluconate, calcium glycerophosphate, calcium hydroxide, calcium lactate, calcium phthalate, calcium phosphates, calcium succinate, calcium tartrate, calcium propionate, dibasic sodium phosphate, dipotassium hydrogen thosphate, dipotassium phosphate, disodium hydrogen phosphate, disodium succinate, potassium succinate, potassium tartrate, sodium acetate, sodium borate, sodium carbonate, sodium citrate, sodium gluconate, sodium hydrogen phosphate, sodium hydroxide, sodium lactate, sodium phthalate, sodium phosphate and sodium polyphosphate. Preferably used Buffering agent is sodium bicarbonate.

The concentration of buffering agents used in the sublingual tablet is from 2% to 15% (w/w), more preferably 5% to 10% (w/w) of the total weight of the composition.

Lubricant used in the present invention are selected from and not limited to sodium stearyl fumarate, sodium oleate, sodium stearate, sodium chloride, stearic acid, magnesium stearate, corn starch, sodium benzoate, light mineral oil, sodium acetate, calcium stearate and other metal stearates, talc, alkyl sulfate, wax, glyceride, PEG, glyceryl behenate, sodium acetate, colloidal silica, hydrogenated vegetable oil, polyethylene glycol, sodium benzoate, but are not limited thereto. Preferably used lubricant is magnesium stearate.

The concentration of lubricant used in the sublingual tablet is from 0.1% to 3% (w/w), more preferably 0.1% to 2% (w/w) of the total weight of the composition.

Solvent used in the present invention are selected from and not limited to absolute alcohol, isopropanol and propanol. Preferably used solvent is absolute alcohol.

The final weight of the sublingual tablet of the present invention is from 50 mg to 500 mg.

The present invention is illustrated in detail but not limiting to, the following examples. It will be apparent to those skilled in the art that many modifications, both to materials and methods, may be practiced without departing from the scope of the invention.

### EXAMPLES

### Example 1

| | | | Concentration |
|---|---|---|---|
| S.No | Ingredients | Mg/Unit | (% w/w) |
| 1. | Levothyroxine Sodium | 0.02755 | 0.03 |
| 2. | Mannitol + Starch (Pearlitol Flash) | 76.67240 | 76.67 |
| 3. | Silicified Microcrystalline cellulose | 10.000 | 10.00 |
| 4. | Sodium Bicarbonate | 6.500 | 6.50 |
| 5. | Croscarmellose Sodium | 2.000 | 2.00 |
| 6. | Butylated hydroxyanisole | 0.200 | 0.20 |
| 7. | Sodium alginate | 3.000 | 3.00 |
| 8. | Sodium lauryl sulphate | 1.000 | 1.00 |
| 9. | Magnesium stearate | 0.600 | 0.60 |
| | Total weight | 100.00 | 100.00 |

### Manufacturing Process:

Levothyroxine Sodium (API) co-sifted with Sodium Bicarbonate and Pearlitol Flash and geometrically mixed in the Rapid Mixer Granulator. Other diluents like Silicified Microcrystalline cellulose, Croscarmellose sodium, Butylated Hydroxy anisole, Sodium alginate and Sodium Lauryl sulphate sifted and mixed in RMG. Lubricated with Magnesium stearate. Compressed to tablets with 100 mg target weight.

### Physical Parameters of the Composition of Example 1

Physical Parameters

| | 6.3 mm FFBE with |
|---|---|
| Tooling | Breakline on one side |
| Weight (mg) | 99.17-101.06 |
| Thickness (mm) | 2.37-2.45 |
| Hardness (Kp) | 1.5-2.3 |
| % Friability | 0.18 |
| DT (Mints) | 17 sec-30 sec |
| Wetting Time (sec.) | more than 5 minutes |
| Water Absorption Ratio | 25.71% |
| Invitro Dispersion Time | 90 sec |
| (Sec.) | |

### Chemical Parameters of the Composition of Example 1

Analytical Parameters

| Assay (%) | | | 98.65 | |
|---|---|---|---|---|
| Related | Liothyronine | NMT 2.0% | 0.028 | |
| Substances | T3 Acetic acid | NMT 1.0% | ND | |
| | T4-Formc acid N- | NMT 1.0% | ND | |
| | Methylamine | | | |
| | IMP-B | NMT 1.0% | ND | |
| | IMP-D | NMT 1.0% | 0.074 | |
| | IMP-F | NMT 1.0% | ND | |
| | IMP-G | NMT 1.0% | ND | |
| | IMP-H | NMT 1.0% | ND | |
| | IMP-I | NMT 1.0% | 0.082 | |
| | Max Unknown | NMT 1.0% | 0.071 @ 1.68 RRT | |
| | Total | NMT 4.0% | 0.26 | |
| Dissolution | | | % | % |

| @ | Time in min | Release | RSD |
|---|---|---|---|
| 0.01N HCl + | 5 min | 63.2 | 16.6 |
| 0.2% SLS | 10 min | 84.0 | 5.5 |
| Buffer, USP-II, | 15 min | 88.5 | 3.2 |
| 500 mL, 50 rpm | 30 min | 90.6 | 3.0 |

### Example 2

| | | | Concentration |
|---|---|---|---|
| S.No | Ingredients | Mg/Unit | (% w/w) |
| 1. | Levothyroxine Sodium | 0.02755 | 0.03 |
| 2. | Mannitol | 76.67240 | 76.67 |
| 3. | Silicified MCC | 10.000 | 10.00 |
| 4. | Sodium Bicarbonate | 6.500 | 6.50 |
| 5. | Croscarmellose Sodium | 2.000 | 2.00 |
| 6. | Butylated hydroxy anisole | 0.200 | 0.20 |
| 7. | Sodium alginate | 3.000 | 3.00 |
| 8. | Glyceryl Monostearate | 1.000 | 1.00 |
| 9. | Magnesium stearate | 0.600 | 0.60 |
| | Total weight | 100.00 | 100.00 |

### Manufacturing Process:

Levothyroxine Sodium (API) co-sifted with Sodium Bicarbonate and Pearlitol Flash and geometrically mixed in the Rapid Mixer Granulator. Other diluents like Silicified Microcrystalline cellulose, Croscarmellose sodium, Butylated Hydroxyanisole, Sodium alginate and Glyceryl Mono stearate sifted and mixed in RMG. Lubricated with Magnesium stearate. Compressed to tablets with 100 mg target weight.

### Physical Parameters of the Composition of Example 2

Physical Parameters

| | 6.3 mm FFBE with | |
|---|---|---|
| Tooling | Breakline on one side | |
| Weight (mg) | 100.20-101.83 | |
| Thickness (mm) | 2.39-2.45 | |
| Hardness (Kp) | 1.4-2.5 | |
| % Friability | 0.28 | |
| DT (Mints) | 13-21 | sec |
| Wetting Time (sec.) | more than 5 minutes | |
| Water Absorption Ratio | 61.65% | |
| In vitro Dispersion Time | 23 | sec |

(Sec.)

### Chemical Parameters of the Composition of Example 2

Analytical Parameters

| Assay (%) | | | 99.8 |
|---|---|---|---|
| Related | Liothyronine | NMT 2.0% | 0.048 |
| Substances | T3 Acetic acid | NMT 1.0% | 0.332 |
| | T4-Formc acid | NMT 1.0% | ND |
| | N-Methylamine | | |
| | IMP-B | NMT 1.0% | ND |
| | IMP-D | NMT 1.0% | 0.061 |
| | IMP-F | NMT 1.0% | ND |
| | IMP-G | NMT 1.0% | ND |
| | IMP-H | NMT 1.0% | ND |
| | IMP-I | NMT 1.0% | 0.081 |
| | Max Unknown | NMT 1.0% | 0.059 @ 1.60 RRT |
| | Total | NMT 4.0% | 0.58 |

| | | % | % |
|---|---|---|---|
| Dissolution @ | Time in min | Release | RSD |
| 0.01N HCl + 0.2% | 5 min | 70.8 | 8.0 |
| SLS Buffer, USP-II, | 10 min | 89.9 | 4.5 |
| 500 mL, 50 rpm | 15 min | 92.7 | 4.6 |
| | 30 min | 94.1 | 4.3 |

### Example 3

| | | | Concentration |
|---|---|---|---|
| S.No | Ingredients | Mg/Unit | (% w/w) |
| 1. | Levothyroxine Sodium | 0.33058 | 0.33 |
| 2. | Mannitol | 59.86940 | 59.87 |
| 3. | Silicified MCC | 25.000 | 25.00 |
| 4. | Croscarmellose Sodium | 10.000 | 10.00 |
| 5. | Butylated hydroxy anisole | 0.200 | 0.20 |
| 6. | Sodium alginate | 3.000 | 3.00 |
| 7. | Sodium lauryl sulphate | 1.000 | 1.00 |
| 8. | Magnesium stearate | 0.600 | 0.60 |
| | Total weight | 100.00 | 100.00 |

### Manufacturing Process:

Levothyroxine Sodium (API) co-sifted with Pearlitol Flash and geometrically mixed in the Rapid Mixer Granulator. Other diluents like Silicified Microcrystalline cellulose, Croscarmellose sodium, Butylated Hydroxyanisole, Sodium alginate and Sodium Lauryl sulphate sifted and mixed in RMG. Lubricated with Magnesium stearate. Compressed to tablets with 100 mg target weight.

### Physical Parameters of the Composition of Example 3

Physical Parameters

| | 6.3 mm FFBE with | |
|---|---|---|
| Tooling | Breakline on one side | |
| Weight (mg) | 99.70-101.03 | |
| Thickness (mm) | 2.56-2.70 | |
| Hardness (Kp) | 0.5-1.2 | |
| % Friability | 0.58 | |
| DT (Mints) | 22-30 | sec |
| Wetting Time (sec.) | more than 5 minutes | |
| Water Absorption Ratio | 78.41% | |
| Invitro Dispersion Time | 64 | sec |

(Sec.)

### Chemical Parameters of the Composition of Example 3

Analytical Parameters

| Assay (%) | | | 99.4 |
|---|---|---|---|
| Related | Liothyronine | NMT 2.0% | 0.009 |
| Substances | T3 Acetic acid | NMT 1.0% | ND |
| | T4-Formc acid | NMT 1.0% | ND |
| | N-Methylamine | | |
| | IMP-B | NMT 1.0% | ND |
| | IMP-D | NMT 1.0% | 0.027 |
| | IMP-F | NMT 1.0% | 0.093 |
| | IMP-G | NMT 1.0% | ND |
| | IMP-H | NMT 1.0% | 0.075 |
| | IMP-I | NMT 1.0% | 0.059 |
| | Max Unknown | NMT 1.0% | 0.070 @ 1.62 RRT |
| | Total | NMT 4.0% | 0.42 |

| | | % | % |
|---|---|---|---|
| Dissolution @ | Time in min | Release | RSD |
| 0.01N | 5 min | 51.8 | 11.5 |
| HCl + 0.2% SLS | 10 min | 75.5 | 11.5 |
| Buffer, USP-II, | 15 min | 78.3 | 11.7 |
| 500 mL, 50 rpm | 30 min | 85.7 | 13.0 |

### Example 4

| | | | Concentration |
|---|---|---|---|
| S.No | Ingredients | Mg/Unit | (% w/w) |
| 1. | Levothyroxine Sodium | 0.33058 | 0.33 |
| 2. | Mannitol | 59.86940 | 59.87 |
| 3. | Silicified MCC | 25.000 | 25.00 |
| 4. | Croscarmellose Sodium | 10.000 | 10.00 |
| 5. | Butylated hydroxy anisole | 0.200 | 0.20 |
| 6. | Sodium alginate | 3.000 | 3.00 |
| 7. | Glyceryl Monostearate | 1.000 | 1.00 |
| 8. | Magnesium stearate | 0.600 | 0.60 |
| | Total weight | 100.00 | 100.00 |

### Manufacturing Process:

Levothyroxine Sodium (API) co-sifted with Pearlitol Flash and geometrically mixed in the Rapid Mixer Granulator. Other diluents like Silicified Microcrystalline cellulose, Croscarmellose sodium, Butylated Hydroxyanisole, Sodium alginate and Sodium Lauryl sulphate sifted and mixed in RMG. Lubricated with Magnesium stearate. Compressed to tablets with 100 mg target weight.

### Physical Parameters of the Composition of Example 4

Physical Parameters

| | 6.3 mm FFBE with | |
|---|---|---|
| Tooling | Breakline on one side | |
| Weight (mg) | 99.53-101.06 | |
| Thickness (mm) | 2.52-2.60 | |
| Hardness (Kp) | 0.9-1.8 | |
| % Friability | 0.33 | |
| DT (Mints) | 24-30 | sec |
| Wetting Time (sec.) | more than 5 minutes | |
| Water Absorption Ratio | 106.94% | |
| In vitro Dispersion Time | 43.66 | sec |

(Sec.)

### Chemical Parameters of the Composition of Example 4

Analytical Parameters

| Assay (%) | | | 98.3 |
|---|---|---|---|
| Related Substances | Liothyronine | NMT 2.0% | 0.047 |
| | T3 Acetic acid | NMT 1.0% | 0.142 |
| | T4-Formc acid | NMT 1.0% | ND |
| | N-Methylamine | | |
| | IMP-B | NMT 1.0% | ND |
| | IMP-D | NMT 1.0% | 0.077 |
| | IMP-F | NMT 1.0% | ND |
| | IMP-G | NMT 1.0% | ND |
| | IMP-H | NMT 1.0% | 0.026 |
| | IMP-I | NMT 1.0% | 0.037 |
| | Max Unknown | NMT 1.0% | 0.068 @ 1.61 RRT |
| | Total | NMT 4.0% | 0.42 |

| | | % | % |
|---|---|---|---|
| Dissolution @ | Time in min | Release | RSD |
| 0.01N HCl + 0.2% | 5 min | 41.4 | 19.1 |
| SLS Buffer, USP-II, | 10 min | 61.6 | 18.7 |
| 500 mL, 50 rpm | 15 min | 67.9 | 16.8 |
| | 30 min | 71.3 | 11.2 |

### Example 5

| | | | |
|---|---|---|---|
| | | | Concentration |
| S.No | Ingredients | Mg/Unit | (% w/w) |

Intra granular Portion

| | | | |
|---|---|---|---|
| 1. | Levothyroxine Sodium | 0.33058 | 0.33 |
| 2. | Mannitol | 59.86940 | 59.87 |
| 3. | Sodium lauryl sulphate | 1.000 | 1.00 |
| 4. | Croscarmellose Sodium | 10.000 | 10.00 |
| 5. | Butylated hydroxy anisole | 0.200 | 0.20 |
| 6. | Absolute alcohol | QS | QS |
| | Extra-granular portion | | |
| 7. | Silicified MCC | 25.000 | 25.00 |
| 8. | Sodium alginate | 3.000 | 3.00 |
| 9. | Magnesium stearate | 0.600 | 0.60 |
| | Total | 100 | 100 |

### Manufacturing Process:

Preparation of the Granulating Solvent (12.0% wow). Dissolved BHA in absolute alcohol. Sifted small portion of Mannitol through #40 mesh loaded into 2 L RMG and mixed for 01 mi at 60 RPM Impeller speed. Mannitol (Pearlitol 160C) and API co-sifted through 40 # and added above step. Sodium Lauryl sulphate and Croscarmellose Sodium were co-sifted through #40 mesh and added to above step; mixed for 10 mints at 60 RPM impeller speed. Above mix granulated with granulating solvent; dried; milled; calculated for % yield. Silicified MCC and Sodium alginate was co-sifted through #40 mesh and added to granular portion in the RMG and mixed for 10 mints at 60 RPM impeller speed. Magnesium stearate was sifted through #60 mesh and added to step 7 and mixed for 03 mints at 60 RPM Impeller speed.

### Physical Parameters of the Composition of Example 5

Physical Parameters

| | 6.3 mm FFBE with | |
|---|---|---|
| Tooling | Breakline on one side | |
| Weight (mg) | 99.73-101.91 | |
| Thickness (mm) | 2.48-2.56 | |
| Hardness (Kp) | 0.6-1.2 | |
| % Friability | 66 | |
| DT (Mints) | 21-25 | sec |
| Wetting Time (sec.) | 04 minutes | |
| Water Absorption Ratio | 109.23% | |
| Invitro Dispersion Time | 28 | sec |

(Sec.)

### Chemical Parameters of the Composition of Example 5

Analytical Parameters

| Assay (%) | | | 96.0 |
|---|---|---|---|
| Related | Liothyronine | NMT 2.0% | 0.009 |
| Substances | T3 Acetic acid | NMT 1.0% | ND |
| | T4-Formc acid | NMT 1.0% | ND |
| | N-Methylamine | | |
| | IMP-B | NMT 1.0% | 0.022 |
| | IMP-D | NMT 1.0% | 0.036 |
| | IMP-F | NMT 1.0% | 0.080 |
| | IMP-G | NMT 1.0% | ND |
| | IMP-H | NMT 1.0% | 0.043 |
| | IMP-I | NMT 1.0% | 0.054 |
| | Max Unknown | NMT 1.0% | 0.126 @ 0.79 RRT |
| | Total | NMT 4.0% | 0.56 |

| | | % | % |
|---|---|---|---|
| Dissolution @ | Time in min | Release | RSD |
| 0.01N HCl + 0.2% | 5 min | 60.7 | 14.0 |
| SLS Buffer, USP-II, | 10 min | 78.1 | 7.6 |
| 500 mL, 50 rpm | 15 min | 82.5 | 7.8 |
| | 30 min | 85.7 | 6.7 |

### Example 6

| | | | Concentration |
|---|---|---|---|
| S.No | Ingredients | Mg/Unit | (% w/w) |
| 1. | Levothyroxine sodium | 0.3 | 0.3 |
| 2. | Mannitol | 78 | 78 |
| 3. | Croscarmellose sodium | 2 | 2 |
| 4. | Butylated hydroxyanisole | 0.2 | 0.2 |
| 5. | Sodium lauryl sulfate | 1 | 1 |
| 6. | Absolute alcohol | qs | qs |
| | Total weight of Blend | 81.5 | 81.5 |
| 7. | Sodium alginate | 3 | 3 |
| 8. | Microcrystalline cellulose | 14.9 | 14.9 |
| 9. | Magnesium stearate | 0.6 | 0.6 |
| | Total | 100 | 100 |

### Manufacturing Process:

Levothyroxine sodium, Sodium lauryl sulfate and Butylated hydroxyanisole (in poly bag) were mixed geometrically. Obtained blend was mixed with croscarmellose sodium and then with mannitol. Obtained blend was granulated with absolute alcohol (96%) using RMG or FBC and dried in Fluidized bed dryer (FBD). Dry blend was taken in rapid mixer granulator (RMG). Sodium alginate powder (fine grade) was added. Microcrystalline cellulose was added and blended for 10-15 min. Obtained blend was lubricated with magnesium stearate for 5-10 min. Lubricated blend was compressed into tablets using round punches. Tablets were packed in HDPE bottle/blister pack with Stabilox and desiccant (if required).

### Example 7

| | | | Concentration |
|---|---|---|---|
| S.No | Ingredients | Mg/Unit | (% w/w) |
| 1. | Levothyroxine sodium | 0.3 | 0.3 |
| 2. | Pearlitol flash | 80 | 80 |
| 3. | Croscarmellose sodium | 2.2 | 2.2 |
| 4. | Butylated hydroxyanisole | 0.2 | 0.2 |
| 5. | Sodium lauryl sulfate | 1.0 | 1.0 |
| 6. | Sodium alginate | 3 | 3 |
| 7. | Microcrystalline cellulose | 13.3 | 13.3 |
| 8. | Magnesium stearate | 0.6 | 0.6 |
| | Total | 100 | 100 |

### Manufacturing Process:

Levothyroxine sodium, Sodium lauryl sulfate and Butylated hydroxyanisole (in poly bag) were mixed geometrically. Obtained blend was mixed with croscarmellose sodium and then with Pearlitol Flash for 10 minutes. Sodium alginate powder (fine grade) was added. Microcrystalline cellulose was added and blended for 10-15 min. Obtained blend was lubricated with magnesium stearate for 5-10 min. Lubricated blend was compressed into tablets using round punches and packed in HDPE bottle/blister pack with Stabilox and desiccant (if required).

### Example 8

| | | | Concentration |
|---|---|---|---|
| S.No | Ingredients | Mg/Unit | (% w/w) |
| 1. | Levothyroxine sodium | 0.3 | 0.3 |
| 2. | Pearlitol flash | 80 | 80 |
| 3. | Croscarmellose sodium | 2.2 | 2.2 |
| 4. | Butylated hydroxyanisole | 0.2 | 0.2 |
| 5. | Glyceryl monostearate (GMS) | 1.0 | 1.0 |
| 6. | Sodium alginate | 3 | 3 |
| 7. | Microcrystalline cellulose | 13.3 | 13.3 |
| 8. | Magnesium stearate | 0.6 | 0.6 |
| | Total weight | 100 | 100 |

### Manufacturing Process:

Levothyroxine sodium, glyceryl monostearate and butylated hydroxyanisole (in poly bag) were mixed geometrically. Obtained blend was mixed with croscarmellose sodium and then with Pearlitol Flash for 10 minutes. Sodium alginate powder (fine grade) was added. Microcrystalline cellulose was added and blended for 10-15 min. Obtained blend was lubricated with magnesium stearate for 5-10 min. Lubricated blend was compressed into tablets using round punches and packed in HDPE bottle blister pack with Stabilox and desiccant (if required).

1: A sublingual tablet composition comprising:
a) 0.01% to 3% w/w of Levothyroxine sodium,
b) 65% to 95% w/w of fillers or diluents,
c) 1% to 15% w/w of superdisintegrants,
d) 0.1% to 3% w/w of antioxidants,
e) 0.1% to 3% w/w of surfactants,
f) 1% to 5% w/w of stabilizers,
g) 0.1% to 3% w/w of lubricant, and optionally
h) 0.1% to 10% w/w of other pharmaceutically acceptable excipients.

2: The composition as claimed in claim 1, wherein said fillers or diluents are selected from mannitol, Pearlitol flash (co-processed mannitol and starch), microcrystalline cellulose, silicified microcrystalline cellulose, dibasic calcium phosphate, powdered cellulose, tribasic calcium phosphate, calcium carbonate, calcium sulfate, dextran, dextrin, dextrose, fructose, kaolin, lactose, sorbitol, starch, pregelatinized starch, sucrose, xylitol and lactose.

3: The composition as claimed in claim 1, wherein said superdisintegrants are selected from sodium starch glycolate and croscarmellose sodium.

4: The composition as claimed in claim 1, wherein said antioxidants are selected from ascorbic acid, citric acid, butylated hydroxyanisole, butylated hydroxytoluene (BHT), potassium metabisulfite, propyl gallate and tocopherol.

5: The composition as claimed in claim 1, wherein said surfactants are selected from sodium lauryl sulfate, glyceryl monostearate and poloxamers (polyoxyethylene and polyoxypropylene copolymers), natural or synthetic lecithin, esters of sorbitan and fatty acids.

6: The composition as claimed in claim 1, wherein said stabilizers are selected from sodium alginate, agar, alginic acid and alginates and carrageenan calcium.

7: The composition as claimed in claim 1, wherein said lubricants are selected from sodium stearyl fumarate, sodium oleate, sodium stearate, sodium chloride, stearic acid, magnesium stearate, calcium stearate and other metal stearates, talc, alkyl sulfate, wax, glyceride, colloidal silica and hydrogenated vegetable oil.

8: The composition as claimed in claim 1, wherein said sublingual tablet composition comprising:
a) 0.01% to 3% w/w of Levothyroxine sodium,
b) 65% to 95% w/w of fillers or diluents selected from mannitol or combination of mannitol and starch, microcrystalline cellulose and silicified microcrystalline cellulose or combinations thereof,
c) 1% to 15% w/w of croscarmellose sodium,
d) 0.1% to 3% w/w of butylated hydroxyanisole,
e) 0.1% to 3% w/w of sodium lauryl sulfate or glyceryl monostearate,
f) 1% to 5% w/w of sodium alginate,
g) 0.1% to 3% w/w of magnesium stearate and optionally
h) 0.1% to 10% w/w of other pharmaceutically acceptable excipients selected from buffering agents and solvents.

9: A process for preparing sublingual tablet as claimed in claim 1, the process comprising steps of:
a) mixing active ingredient geometrically with fillers/diluents,
b) mixing obtained blend with other excipients,
c) lubricating the obtained blend with lubricant,
d) compressing the lubricated blend into tablet, and
e) packing obtained sublingual tablet.

10: A process for preparing sublingual tablet as claimed in claim 1, the process comprising steps of:
a) mixing active ingredient geometrically with surfactant and antioxidant,
b) mixing obtained blend with superdisintegrant and then with fillers/diluents,
c) granulating the obtained blend with solvent using RMG or Fluidized Bed Coater (FBC) followed by drying in Fluidized bed dryer (FBD),
d) adding stabilizing agent, fillers/diluents and blending for 5-10 min using rapid mixer granulator (RMG),
e) lubricating the obtained blend with lubricant for 5-10 min,
f) compressing the lubricated blend into, and
g) packing obtained sublingual tablet in HDPE bottle/blister pack.
